(19) United States
(12) Patent Application Publication
(10) Pub. No.: US 2017/0266899 A1
(43) Pub. Date: Sep. 21, 2017

(54) Breast prosthesis having a closure stopper for a filling passage

(71) Applicant: Amoena Medizin-Orthopaedie-Technik GmbH, Raubling (DE)

(72) Inventor: Nils STELTER, Frasdorf (DE)

(21) Appl. No.: 15/464,126

(22) Filed: Mar. 20, 2017

(30) Foreign Application Priority Data
Mar. 21, 2016 (DE) 10 2016 003 448.3

(51) Int. Cl. B29D 22/00; B29C 65/48; A61F 2/12

(52) U.S. Cl. CPC B29D 22/00; A61F 2/12; B29C 65/4815; B29K 2083/005

(57) ABSTRACT

The invention relates to a method of manufacturing a breast prosthesis having a shell-like body that has a film bag having a filling, wherein the method comprises the following steps: welding and/or adhering plastic films while leaving a region free to form a film bag having a filling passage; injecting a flowable filler material through the filling passage into the film bag; and welding and/or adhering the plastic films in the region of the filling passage to close the film bag, wherein a flowable sealing compound is injected that comes to lie between the filling passage and the filler material and forms a stopper after the injection of the filler material and before the closing of the film bag.

The invention relates to a method of manufacturing a breast prosthesis having a shell-like body that has a film bag having a filling. The invention further relates to a breast prosthesis that can be manufactured using such a method.

Breast prostheses comprising film bags having a flowable filler material have the problem that the filler material exerts an elevated pressure in wear and in quality inspection and thus exerts an increased mechanical strain on the weld seams and/or adhesive seams of the film bag. The weak point in this respect is the filling passage that is closed at the end of the manufacturing process and that does always withstand these strains. For on the introduction of the filler material, residues often accumulate in the filling passage that make a welding during a thermal process such as a furnace process more difficult or only allow it in an insufficient manner. A subsequent welding or adhesion can improve this situation, but cannot always solve it satisfactorily and permanently.

It is the object of the invention to provide a method of manufacturing a breast prosthesis with which these disadvantages are overcome.

Against this background, the invention relates to a method of manufacturing a breast prosthesis having a shell-like body that has a film bag having a filling, wherein the method comprises the following steps: (a) welding and/or adhering plastic films while leaving a region free to form a film bag having a filling passage; (b) injecting a flowable filler material through the filling passage into the film bag; and (c) welding and/or adhering the plastic films in the region of the filling passage to close the film bag. Provision is made in accordance with the invention that a flowable sealing compound is injected that comes to lie between the filling passage and the filler material and forms a stopper after the injection of the filler material and before the closing of the film bag.

Both the filler material and the sealing compound are constituted such that they can be injected into the film bag. They are therefore flowable and preferably viscous.

In an embodiment, the sealing compound is injected such that it completely covers the filling passage internally with respect to the filler material. In an embodiment, the sealing compound is injected such that it expands in cap shape, starting from the injection opening and its environment, into the film bag and displaces the filler material in so doing.

In an embodiment, the sealing compound is configured such that it can be solidified to form an elastic sealing material.

In an embodiment, the sealing compound is a curable silicone and is preferably liquid silicone. In another embodiment, the sealing compound is a hot melt adhesive that is injected at an elevated injection temperature in a liquid state. In yet another embodiment, the sealing compound is a chemically hardening and silicone-free plastic adhesive. Examples include polymerization adhesives, for example acrylate-based, polycondensation adhesives, for example imide-based or sulfide-based, and polyaddition adhesives, for example epoxy-based or urethane-based.

In an embodiment, the method furthermore comprises the step of solidification and preferably curing of the sealing compound. It can in this respect, for example, be a thermal curing and/or a radiation curing. A rubber-elastic stopper is thus formed that prevents a pressing of the filler material onto the weld seam and/or adhesive seam in the region of the former filling passage.

A portion of the sealing compound can still be located between the plastic films in the region of the filling opening during the welding or adhering of the plastic films and can serve as a welding aid or as a thermally activated adhesive.

In an embodiment, the filler material is also formed such that it can be solidified to form a rubber-elastic material. In an embodiment, the filler material is liquid silicone.

In an embodiment, the filler material is formed such that it cannot be solidified or cannot be completely solidified. This can result in the formation of shear thinning compounds having a flow limit. Examples include low-cured silicone gels. In this embodiment, the stopper can prove to be particularly advantageous since the pressure on the weld seam or adhesive seam can in particular be large in the region of the former filling opening with flowable fillings.

Against the initially named background, the invention further relates to a method of manufacturing a breast prosthesis having a shell-like body that has a film bag having a filling, wherein the method comprises the following steps: (a) welding and/or adhering plastic films while leaving a region free to form a film bag having a filling passage; (b) injecting a flowable and curable filler material through the filling passage into the film bag; and (c) welding and/or adhering the plastic films in the region of the filling passage to close the film bag. Provision is made in accordance with the invention that, after the injection of the filler material and before the closing of the film bag, a curing agent is injected that mixes with the filler material in the region of the filling passage or that a curing agent is externally applied to the plastic films in the region of the filling passage.

This requires that a curable material is used as the filler material. Either a curing agent is therefore introduced after the filling and before the, for example, thermal curing process for the filler material via the filling passage to achieve higher curing toward the stopper or a curing agent is applied from the outside to be able to diffuse through the film during the thermal process such as the furnace process and to be able likewise to effect a stopper formation by additional curing.

The curing agent is a compound having two or more reactive groups that can react with corresponding reactive groups of the filler material.

The curing agent or alternatively the injected carrier composition of the curing agent is also flowable and preferably viscous.

In an embodiment, the method furthermore comprises the step of curing the filler material. It can in this respect, for example, be a thermal curing and/or a radiation curing. Due to the presence of the curing agent close to the injection point, the degree of curing is higher there and a rubber-elastic stopper is formed that prevents a pressing of the filler material onto the weld seam or the adhesive seam in the region of the former filling passage.

The welding of the plastic films can comprise a thermal welding, for example. Other methods such as ultrasound welding are also conceivable.

Against the initially named background, the invention furthermore relates to a breast prosthesis having a shell-like body that has a film bag having a filling, wherein the film bag comprises plastic films connected along the weld seam and/or adhesive seam and wherein a stopper of a sealing material is located between the filling and a part section of the weld seam or adhesive seam.

In an embodiment, the sealing material is a non-flowable elastic plastic material, for example silicone rubber. The non-flowable elastic plastic material can, however, also be silicone-free.

In an embodiment, the stopper adjoins a part section of the weld seam or adhesive seam in cap form.

In an embodiment, the filling is a shear thinning compound having a flow limit, for example a low-cured silicone gel. In another embodiment, the filling is a rubber-elastic compound, for example a two-component silicone rubber compound.

Provision is made in an embodiment that the film bag is formed by two plastic films welded and/or adhered along the common periphery.

The basic idea of the present invention is therefore to close the pressure-susceptible filling passage with an elastic stopper that cushions the pressures emanating from the filler material and that reduces the loads incoming at the weld seam or adhesive seam formed in the injection region to a minimum.

The stopper results from a flowable mass that presses the residues of the filler material out of the filling passage during the injection and in so doing forms a cap shape. This material is elastically cured or solidified during a thermal process such as a furnace process and forms a good film adhesion as an anchorage. The critical filling passage is thus well protected against bursting caused by pressing and prevents an escape of the filler material.

Further details and advantages of the invention will be described with reference to the embodiment shown in the Figures. There are shown in the Figures:

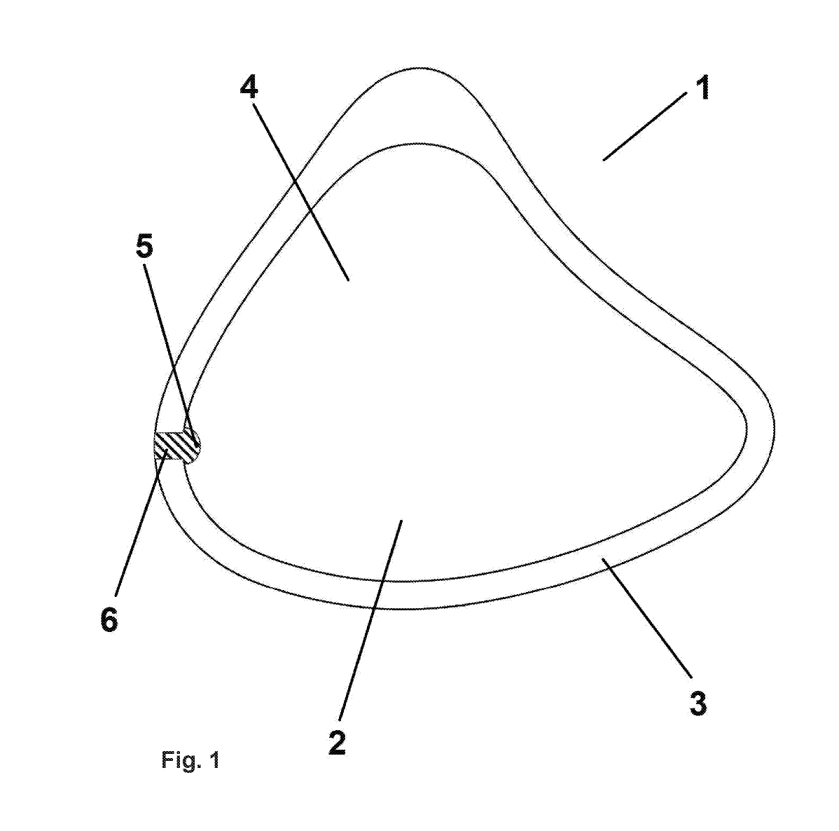

FIG. 1: a plan view of a film bag of a breast prosthesis in accordance with the invention; and

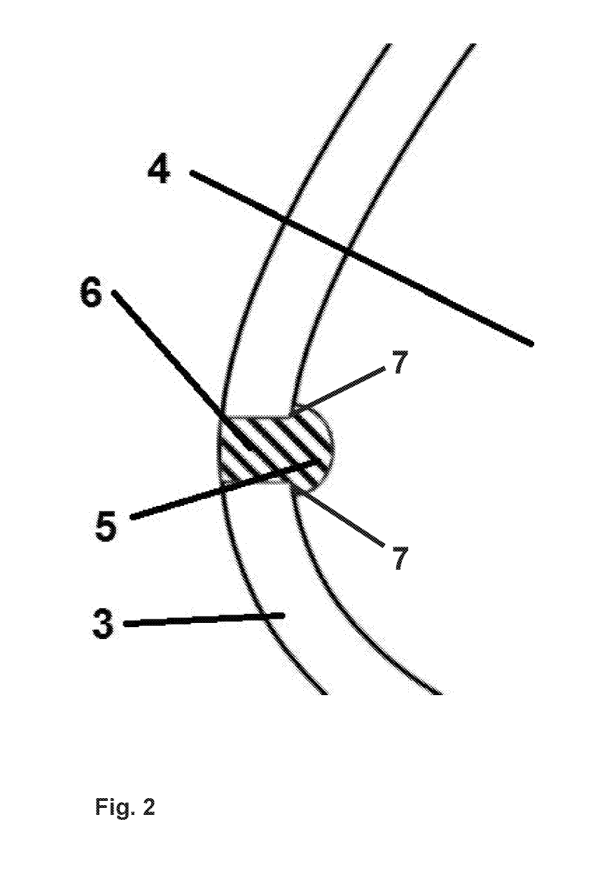

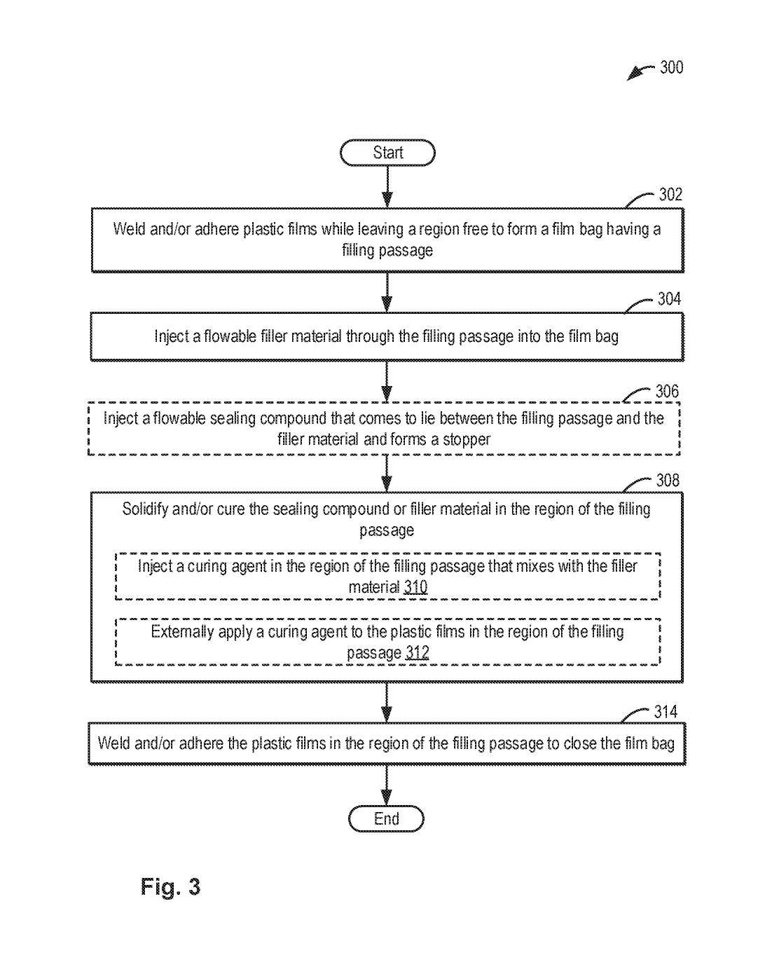

FIG. 2: an enlarged representation of the region of the closed filling opening with a stopper.

The Figures show a shell-like film bag of a breast prosthesis in accordance with the invention in a plan view. The film bag is generally marked by the reference numeral 1. It forms a breast prosthesis in addition to at least one further shell-like film bag that can adjoin in the form of a sandwich as well as, optionally, further elements such as textile inserts.

The film bag 1 is formed by two congruent film pieces 2 of a thermoplastic plastic film that are welded along the common peripheral margin and form a weld seam 3. A low-cured silicone gel is located as a filler material, that is plastically deformable, in the chamber 4 that is bounded by the plastic films 2. The film bag 1 shown therefore forms the part of a breast prosthesis that can deform plastically and thus makes it possible that the breast prosthesis can be matched to the shape of the scar tissue.

A stopper 5 of cured silicone rubber contacts the weld seam 3 in a small marginal region of the chamber 4. The stopper extends, starting from a section 6 of the weld seam at which the filling opening was located during the manufacturing process of the film bag 1, into the interior of the chamber 4 in a cap shape. The material of the stopper is also present in the region 6 of the weld seam and serves as a welding aid or as a thermally activated adhesive in this region.

On the manufacture of the shown film bag 1, the two plastic films 2 are first welded along the common periphery while leaving free the region 6 to form a film bag. The filling opening for the chamber 4 is located in the region left free. Subsequently, a flowable filler material is injected into the chamber 4 through the filling passage. A further injection process subsequently takes place in which a small quantity of a curable sealing compound of liquid silicone is injected that later forms the stopper. The plastic films 2 are subsequently also welded in the region 6 of the filling passage. The liquid silicone of the stopper is thermally activated in this process and serves as a welding aid. The stopper 5 is already vulcanized during this welding closed of the filling passage and also as part of a subsequent furnace process such that it is hardened into a rubber-elastic silicone rubber.

It is thus achieved that the stopper protects the region 6 of the weld seam 3 that represents a weak point for conventional products against the effect of pressure in wear and as part of the quality control and thus the stability and service life of the breast prosthesis is increased.

1. A method of manufacturing a breast prosthesis having a shell-like body that has a film bag having a filling, the method comprising:
welding and/or adhering plastic films while leaving a region free to form a film bag having a filling passage;
injecting a flowable filler material through the filling passage into the film bag; and
welding and/or adhering the plastic films in the region of the filling passage to close the film bag,
wherein a flowable sealing compound is injected that comes to lie between the filling passage and the filler material and forms a stopper after the injection of the filler material and before the closing of the film bag.

2. The method in accordance with claim 1, wherein the sealing compound is configured such that it can be solidified to form an elastic sealing material.

3. The method in accordance with claim 2, wherein the sealing compound is a curable silicone.

4. The method in accordance with claim 2, wherein the sealing compound is a hot melt adhesive that is injected at an elevated injection temperature in a liquid state; or wherein the sealing compound is a chemically hardening and silicone-free plastic adhesive.

5. The method in accordance with claim 1, further comprising solidifying the sealing compound.

6. A method of manufacturing a breast prosthesis having a shell-like body that has a film bag having a filling, wherein the method comprises:
welding and/or adhering plastic films while leaving a region free to form a film bag having a filling passage;
injecting a flowable and curable filler material through the filling passage into the film bag; and
welding and/or adhering the plastic films in the region of the filling passage to close the film bag,
wherein after the injection of the filler material and before the closing of the film bag, a curing agent is injected that mixes with the filler material in the region of the filling passage, or wherein a curing agent is externally applied to the plastic films in the region of the filling passage.

7. A breast prosthesis having a shell-like body that has a film bag having a filling, wherein the film bag comprises plastic films connected along a weld seam and/or adhesive seam,
wherein a stopper of a sealing material is located between the filling and a part section of the weld seam and/or adhesive seam.

8. The breast prosthesis in accordance with claim 7, wherein the sealing material is a non-flowable elastic material.

9. The breast prosthesis in accordance with claim 7, wherein the stopper adjoins the part section of the weld seam and/or adhesive seam in a cap shape.

10. The breast prosthesis in accordance with claim 7, wherein the filling is a shear thinning compound having a flow limit.

11. The method in accordance with claim 3, wherein the sealing compound is liquid silicone.

12. The method in accordance with claim 5, further comprising curing the sealing compound.

13. The method in accordance with claim 8, wherein the sealing material is silicone rubber.

14. The method in accordance with claim 10, wherein the filling is a low-cured silicone gel.
